(19) United States
(12) Patent Application Publication
(10) Pub. No.: US 2011/0271957 A1
(43) Pub. Date: Nov. 10, 2011

(54) Delivery of estrogen and bioequivalents thereof via the pulmonary tract

(75) Inventors: Robert J. Matis, Westerville, OH (US); Bruce McVeety, Dublin, OH (US); David Cline, Dublin, OH (US); Michael D. Triplett, II, New Albany, OH (US)

(73) Assignee: BATTELLE MEMORIAL INSTITUTE, Columbus, OH (US)

(21) Appl. No.: 13/099,426

(22) Filed: May 3, 2011

Related U.S. Application Data
(60) Provisional application No. 61/331,956, filed on May 6, 2010.

(51) Int. Cl. A61M 15/00

(52) U.S. Cl. 128/203.12

(57) ABSTRACT

The instant disclosure relates to methods of providing a composition containing estrogen or a biological equivalent thereof to a subject in need of such treatment, wherein a therapeutically effective amount of the estrogen or biological equivalent thereof is administered to the subject via inhalation of an electrohydrodynamic spray.

### BACKGROUND OF THE INVENTION

Menopause is a natural biological process involving the loss of ovarian follicular function resulting in the permanent cessation of menses. This results in drastically reduced levels of estrogen, documented to fall by up to 80%, which is largely responsible for the gamut of menopausal symptoms and complications. Menopause is not believed to be an estrogen deficiency disease. Regardless, this lack of estrogen and its effects provide the initial rationale for hormone replacement therapy (HRT), not only to relieve symptoms, but also to prevent chronic postmenopausal ailments. Menopause can be spontaneous/natural or induced by medical intervention such as surgery or the iatrogenic ablation of ovarian function (i.e. chemotherapy or pelvic radiation therapy), and also represents the end of natural childbearing.

With the average age of menopause onset being 51 years in the West, coupled with the general aging of the global population, an increasing number of women will spend more than a third of their lives post menopause with the burden of postmenopausal complications becoming heavier in the future. Menopause is rarely an immediate occurrence with the process taking a number of years to complete.

There are a number of symptoms associated with menopausal transition and the early postmenopausal period. While most symptoms are reported to disappear during this early postmenopausal period, a minority of women experience symptoms well into late post-menopause. Estimates suggest that approximately 50-85% of women suffer some kind of menopause-linked symptom, the most common of which are vasomotor symptoms (hot flashes/flushes) and vulvar/vaginal atrophy (involving burning, itching or dryness), but also include insomnia, mood changes, depression and anxiety. Between 10-20% are thought to experience symptoms significant enough to interfere with normal daily functioning. Certain symptoms tend to be associated with different stages of menopausal transition as a result of estrogen loss. The early stages of transition tend to be associated with vasomotor symptoms, mood disturbances, sleep disturbances (such as night sweats and insomnia), forgetfulness and urogenital symptoms. Symptoms common in the intermediate stages of menopausal transition are vaginal atrophy, urinary incontinence and hair loss. However, menopausal symptoms can and do occur at any stage of the transition. During late post-menopause, the majority of women will no longer experience symptoms, however, at this stage women are at increased risk of osteoporosis, heart disease, cancer and dementia.

In the postmenopausal period, there are a number of conditions that women are at an increased risk of developing. These conditions are effectively the result of the hypoestrogenic state that occurs after the menopause, and the disruption of processes normally regulated or affected by the hormone. The three most significant conditions postmenopausal women are at greater risk from are osteoporosis, cardiovascular disease (CVD) and dementia or Alzheimer's disease (AD). With more than 40 million women either going through or past menopause in the US and the aging baby boomer generation expecting to significantly add to this population, the prevalence of these conditions in the older female population will increase. With this demographic change, the cost of treatment and prevention in the postmenopausal population will be an increasingly heavy burden upon healthcare providers.

Although for many women menopause is asymptomatic and associated with little disruption of normal life and well-being, many women experience symptoms—sometimes severe and disabling—that considerably affect their quality of life. Hormone replacement therapy (HRT) is presently believed to be the most effective intervention for management of these quality-of-life symptoms.

The goal of HRT (generally estrogen therapy alone or a combination of estrogen and a progestational agent (E+P)) is the effective relief of symptoms, with few side effects and convenience for women, while providing optimal protective effects in other areas. The practice revolves around the view of menopause as a deficiency disease or condition, rather than a natural stage in a woman's reproductive cycle. As such, the simple replacement of the estrogen and progestin lost at menopause with HRT was long assumed to be the cure to all menopausal ills. While estrogen is well documented to relieve short term menopausal symptoms, long term health benefits were also thought to be had in preventing osteoporosis and potentially cardiovascular disease. However, the Women's Health Initiative (WHI) Study has recently identified increased risks of breast cancer and cardiovascular disease with just over five years of treatment with oral estrogen/progestin. In fact, there have been suggestions of the risk of these conditions with oral hormone replacement for some time.

HRT has been practiced in menopausal and postmenopausal women for around six decades, with conjugated equine estrogen (Premarin®) being one of the first products available. Hormone replacement may begin in women in their forties when menopausal symptoms begin to appear. Treatment duration varies and can last until symptoms subside, anywhere from six months to five years. Longer term treatment is often used in those women who still experience climacteric symptoms well into the postmenopausal stage, while the prevention of osteoporosis is also associated with long term therapy, often ten years or more. The traditional route of administration of HRT has been oral with products in tablet form. Orally delivered hormones, however, must undergo metabolism in the liver (“first pass metabolism”) and as such, estrogens, progestogens and androgens must be administered in relatively high doses. First-pass metabolism inactivates 35-50% of the absorbed estrogen and oral delivery usually results in a peak in estrogen levels 4-8 hours after taking the drug.

While non-oral administration via transdermal patches avoids the first pass effect, thereby allowing for lower estrogen doses to achieve similar hormonal effects, transdermal patches suffer from other drawbacks. The majority of patches are also estrogen only and provide continuous dosing. Most transdermal patches are usually worn for three to four days each, though some may be worn for up to seven days. One of the common side effects reported with hormone patches is skin irritation due to the adhesive used. As an alternative, estrogen creams and gels may be rubbed into the skin of the upper arm or thigh daily, while these formulations are not subject to the irritation that can occur with hormone patches, such treatments may not have the required dose control or therapeutic benefit. Pessaries are also available which may be inserted into the vagina and absorbed through the mucosal layer, as with vaginal creams/gels. While intravaginal delivery allows a more localized effect and is more often used to specifically treat genitourinary symptoms such as vaginal dryness, atrophy and urinary incontinence or urgency, such methods do not generally provide systemic effects of a magnitude necessary for treatment of the body as a whole.

While estrogen replacement therapy is effective for the prevention of osteoporosis in women and has been shown to reduce bone resorption and retard or halt bone loss in postmenopausal women, such therapy is associated with a number of adverse effects. While results from earlier observational studies indicated that estrogen replacement therapy (ERT) or combined estrogen/progestin therapy (HRT) was associated with cardiovascular benefit in postmenopausal women, results from recent controlled studies indicate that hormone therapy does not decrease the incidence of cardiovascular disease. The American Heart Association (AHA), American College of Obstetricians and Gynecologists (ACOG), US Food and Drug Administration (FDA) and manufacturers recommend that hormone therapy not be used to prevent heart disease in healthy women (primary prevention) or to protect women with preexisting heart disease (secondary prevention).

The Women's Health Initiative (WHI) is one of the largest studies of preventive health ever undertaken in the US. It was established in 1991 in collaboration between the National Heart, Lung and Blood Institute and other units of the National Institutes of Health to look at the major causes of death, disability and frailty in postmenopausal women. The initiative spans 15 years and involves 161,000 healthy postmenopausal women aged 50-79 years. There are three key components of the study: a randomized, controlled trial of approaches to prevention; an observational study looking at biological markers and predictors of disease, and a community prevention study that aimed to find ways of persuading women to adopt healthier behaviors. The clinical component consisted of three trials examining the effects of hormone replacement, diet modification and calcium and vitamin D on heart disease, osteoporotic fractures and colorectal cancer risk. The ultimate aim of the WHI is to use prophylactic or intervention strategies to reduce coronary heart disease, breast and colorectal cancer, and osteoporotic fractures among postmenopausal women.

The WHI trials assessing the risks and benefits of HRT involved 27,348 postmenopausal women in total. This trial was split into two parts, an estrogen only arm and an estrogen/progestin combination arm. The estrogen only study involved 10,739 hysterectomized women who were randomized to receive oral estrogen or a placebo. The other study involved 16,608 postmenopausal women with an intact uterus who were randomized to receive oral combination therapy or placebo. The oral estrogen used in both studies was 0.625 mg conjugated equine estrogens (Premarin), combined with 2.5 mg Medroxyprogesterone (sold under the tradename Prempro®) in the estrogen/progestin trial. This particular combination product was chosen because it was the most commonly prescribed combination hormone in the US and previous observational studies suggested that it may be beneficial for women's health. The women were enrolled from 1993-98 and the trials had a planned duration 8.5 years until 2005. An independent panel, the Data and Safety Monitoring Board (DSMB), monitored the women's health throughout the study.

In July 2002, the estrogen/progestin arm of the WHI trials was prematurely halted after 5.2 years when an analysis of the data revealed that the risks outnumbered the benefits among the treated population. The study was due to continue until 2005. This study had 8,506 women using Prempro® and 8,102 taking a placebo. The primary outcome was coronary heart disease with invasive breast cancer as the primary adverse outcome. An index of the risks also included pulmonary embolism, stroke, endometrial cancer, hip fracture and death due to other causes. The study's findings were reported by the writing group for the WHI in the Journal of the American Medical Association. (See, e.g., Risks and Benefits of Estrogen Plus Progestin in Healthy Postmenopausal Women, Principal Results From the Women's Health Initiative Randomized Controlled Trial, Writing Group for the Women's Health Initiative Investigators, JAMA. 2002;288:321-333, See also, Brunner, R L et al; Womens Health Initiative Investigators (2005). “Effects of conjugated equine estrogen on health-related quality of life in postmenopausal women with hysterectomy: results from the Women's Health Initiative Randomized Clinical Trial.” Archives of Internal Medicine 165: 1976-1986.)

The results showed that there were both risks and benefits of estrogen/progestin therapy. It was found that women taking Prempro® were at an increased risk of breast cancer and certain forms of cardiovascular disease and thromboembolic events as compared to women in the placebo group. These risks were found to outweigh the benefits of reduced risk of osteoporotic fractures and colorectal cancer among the treated group compared to placebo.

The results of the Women's Health Initiative (WHI) study have precipitated a dramatic and significant shift in the perception and prescription of HRT, suggesting that the long-term effects of HRT use must be considered. Investigators have proposed that the hypoestrogenic state associated with menopause can adversely affect the target tissues of estrogen action, including the brain, skeleton, integument, and urogenital and cardiovascular systems; nevertheless, the effectiveness of HRT in the prevention of disease remains controversial.

More recently a growing scientific body of evidence published on estradiol delivered by parenteral route (i.e., transdermal patch, intranasal) demonstrates a more favorable safety profile with respect to lipids profiles (cardiac events), clotting factors (thrombosis events, stroke), etc. Hormone replacement products can also be administered topically through transdermal patches and creams, transmucosally through intravaginal creams and gels as well as through intravaginal rings, pessaries and IUDs, nasal sprays, injections and subcutaneous implants. However, each of these forms have drawbacks which limit efficacy and/or alleviation of systemic symptoms.

Accordingly, there remains a need for a hormone replacement therapy that can provide one or more benefits to a subject in need thereof, but which does not have one or more of the problems described above as associated with existing hormone replacement therapies. The disclosed compositions and methods seek to address this need.

### BRIEF SUMMARY OF THE INVENTION

The instant disclosure relates to methods of treating a subject in need of such treatment with an estrogen, for example, estradiol. In one aspect, method utilizes electrohydrodynamic spray to deliver said estrogen to a subject in need of such treatment. In one aspect, devices and compositions related to these methods are disclosed.

### DETAILED DESCRIPTION OF THE INVENTION

### References Cited

1. Newman, S P. Crit Rev Ther Drug Carrier Syst., vol. 10, 1, 65-109, 1993.

2. Zimlich, W C et al. Respiratory Drug Delivery 2000, 241-246, 2000.

3. Newman S P. Eur Respi J3: 495-497, 1990.

4. Hirst P H et al Resp Med, Vol. 95, 720-272, 2001.

5. Pitcairn G R et al., J Aerosol Med, Vol. 18, No. 3, 261-63, 2005.

6. Warren S J and Taylor G. in Respiratory Drug Delivery 1998, 453-455.

7. Duddu S p et al. Pharm Res, Vol. 19, No. 5, 689-95, 2002.

8. Dunbar C et al. Int J. Pharm, Vol. 245, 179-189, 2002.

9. Pitcairn G R et al., In Respiratory Drug Delivery 200249-552.

### DEFINITIONS

For convenience, certain terms employed in the specification, examples and claims are collected here. These definitions should be read in light of the remainder of the disclosure and understood as by a person of skill in the art. Unless defined otherwise, all technical and scientific terms used herein have the same meaning as commonly understood by a person of ordinary skill in the art. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the materials and/or methodologies which are reported in the publications which might be used in connection with the invention. Nothing herein is to be construed as an admission that the invention is not entitled to antedate such disclosure by virtue of prior invention.

As used herein, the articles “a” and “an” refer to one or to more than one (i.e., to at least one) of the grammatical object of the article. By way of example, “an element” means one element or more than one element.

As used herein, the term “bioavailability” is used as is conventionally understood in the art, and generally refers to the percentage of drug that may be detected in the systemic circulation after its administration. Bioavailability may be calculated as described in Shargel, L.; Yu, A. B. (1999). Applied biopharmaceutics & pharmacokinetics (4th ed.). New York: McGraw-Hill. ISBN 0-8385-0278-4, incorporated in this respect by reference.

As used herein, the term “bioequivalent” means a drug that has substantially the same effect on the body, and substantially the same bioavailability, as another drug. In one aspect, a bioequivalent may have a substantially identical chemical structure, but may require a different amount to achieve the same effect.

As used herein, the term “estrogen” refers to a category of hormones, but is largely composed of three compounds: estradiol (E2), estrone (E3) and estriol (E1). Estradiol is the most potent form of estrogen, dominating during the reproductive years, and decreasing at menopause. Estrone is produced in lesser amounts pre-menopause and it has a weaker estrogenic activity than estradiol, however, levels of this compound dominate after menopause. Estriol also has a weaker estrogenic activity and is produced in large amounts during pregnancy.

As used herein, the phrase “effective amount” refers to that amount of a substance that produces some desired local or systemic effect. The effective amount of such substance will vary depending upon the individual and disease condition being treated, the weight and age of the individual, the severity of the disease condition, the manner of administration and the like, which can readily be determined by one of ordinary skill in the art.

As used herein, an “individual” or “subject” means a vertebrate, in one aspect a mammal, in another aspect, a human.

As used herein, the terms “prevent,” “preventing” and “prevention” refer to the prevention of the development, recurrence or onset of a disorder or one or more symptoms thereof resulting from the administration of one or more compounds identified in accordance the methods of the invention or the administration of a combination of such a compound and a known therapy for such a disorder.

As used herein, the term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

As used herein, the phrase “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the individual and disease condition being treated, the weight and age of the individual, the severity of the disease condition, the manner of administration and the like, which can readily be determined by one of ordinary skill in the art.

As used herein, the terms “therapy” and “therapies” refer to any method, protocol and/or agent that can be used in the prevention, treatment, management or amelioration of a disease or disorder or one or more symptoms thereof. Similarly, as used herein, the terms “treat,” “treatment” and “treating” refer to the reduction or amelioration of the progression, severity and/or duration of a disorder or one or more symptoms thereof

As used herein, the term “treatment” (as used in a therapeutic context) means both treatment of current diseases or disorders as well as prevention of or protection from the development of new diseases or disorders, for example.

As used herein, the term “delivery efficiency” means the percent of a metered dose that is emitted using a delivery device.

### DETAILED DESCRIPTION

As described above, over the past ten years, there have been numerous publications associating adverse medical events with oral estrogen use (i.e., synthetic estrogens, conjugated equine estrogens) and their respective metabolites as a consequence of first-pass hepatic metabolism.

These negative findings have not been associated with the use of estradiol transdermal patch products which deliver estradiol directly to the blood via absorption through the skin, and a growing scientific body of evidence published on estradiol delivered by parenteral route (i.e., transdermal patch, intranasal) demonstrate a more favorable safety profile with respect to lipids profiles (cardiac events), clotting factors (thrombosis events, stroke), etc. as compared to oral estrogen delivery. It has been suggested that transdermal delivery may carry fewer risks as the doses required are lower and more closely represent the natural physiology of sex hormones. It also avoids first pass metabolism via the liver. However, transdermal patches suffer from separate drawbacks, namely, continuous delivery and irritation at the site of delivery, among others.

In addition, the WHI estrogen/progestin study only looked at one HRT regimen (CEE 0.625 mg plus MPA 2.5 mg, Wyeth's Premarin). As such, it is unknown whether the results can be applied not only to lower doses of these drugs, but also to alternative delivery methods or to other estrogens or progestogens. Similarly, estradiol has also been proposed as being associated with fewer risks due to the fact that it is closer to the naturally produced hormone.

With respect to intranasal routes, published studies on this route of administration show that the pharmacokinetic (PK) profile has rapid absorption, higher maximum concentration (Cₘₐₓ), rapid elimination, and low conversion to active metabolites (e.g., estrone) because of its avoidance of first-pass hepatic metabolism. The efficacy and safety of estradiol delivered intra-nasally for treatment of menopausal symptoms has been established at doses using 150-300 mcg daily, as compared to transdermal estrogen products which deliver estradiol 50-100 mcg daily, achieving steady estradiol concentrations above pre-treatment baseline for >72 hours (see Devissaguet 1999). Thus, a higher concentration of hormone, when delivered intranasally as compared to transdermally, appears to be necessary. An intranasal product (Aerodiol; Servier Inc, UK) has been marketed in Australia/New Zealand for 5 years; only recently has this product been discontinued from market—due to commercial reasons and not for safety reasons per Servier press release). While nasal sprays may have some advantages, this delivery method can result in side effects such as itching at the site of administration, adverse effects normally associated with HRT such as mastalgia, and may not be suitable for women having persistent rhinorrhoea. Further, with respect to biological efficacy, it is believed that the intranasal delivery route provides only about 25% bioavailability of administered estradiol, resulting in inefficient delivery of the active agent and the need to administer larger amounts of the hormone.

Traditional inhaler-type devices include metered dose inhalers (MDIs), dry powder inhalers (CDPIs) and nebulizers. These devices are well known, and used for a variety of different applications. These devices, in general provide little ability to control for particle size, and as such, where certain particle sizes are desired, such devices cannot generally meet this need. Further, such devices generally provide larger (greater than about 5 μm) particles. Such particles cannot penetrate the lung cavity as profusely, and therefore cannot effectively or rapidly deliver the inhaled agent such that systemic delivery is achieved.

Thus, while the need for safe and effective hormone replacement strategies is a long-felt need, currently available therapy modalities have been generally unsuccessful in meeting this need.

Disclosed herein are methods of providing an estrogen, or bioequivalent thereof, to a subject in need of such treatment, comprising administering a therapeutically effective amount of a carrier comprising the estrogen or bioequivalent thereof by way of the respiratory tract, wherein said administration is achieved via an device capable of producing an electrohydrodynamic spray. In one aspect, the estrogen may be selected from conjugated equine estrogens, estradiol, synthetic conjugated estrogine, micronized estradiol, estropipate, esterified estrogens, salts thereof, and combinations thereof In one aspect, the estrogen may comprise estradiol, such as 17β-estradiol, or a biological equivalent thereof In other aspects, a progesterone or a biological equivalent thereof may be co-administered with the estrogen. In this aspect, the progesterone may be selected from progestin, progesterone, medroxyprogesterone, norethindrone, levonorgestral, norethindrone, norgestimate, drospirenone, salts thereof, or combinations thereof

Electrostatic spraying devices, in which an electric charge is imparted to a liquid before or after it is forced through a nozzle, to provide small, electrostatically charged droplets, are widely known and used for a variety of purposes. These may be distinguished from electrohydrodynamic (EHD) devices, in which an electric charge of sufficient intensity is applied to the fluid to induce aerosolization.

A method of processing liquids using electric fields using the EHD method is described in, for example, WO 2007/094835, US 2008/0308095A1, US 2008/0308580 A1, and GB-A-1569707, all of which are incorporated by reference in their entirety. In this method, liquid issuing from an outlet is subjected to an electric field such that the net electrical charge in the liquid as the liquid emerges into free space or air counteracts the surface tension forces of the liquid and the repulsive forces generated by the like electrical charges result in a cone and jet. Depending upon the liquid formulation, the liquid jet may then break up into liquid droplets, or may, as described in, for example, WO 98/03267, break up to form solid or gel-like particles or may form a continuous fibre which may break up into short lengths (“fibrils”). The products resulting from the electrodynamic method may be collectively referred to as “electrosols”. This electrohydrodynamic method is particularly good at controlling the dimensions of the resultant product and can provide an extremely efficient way of delivering drugs or medicaments to the respiratory system, for example to the pulmonary system.

Furthermore, as described in WO 98/03267 electrohydrodynamic methods may be used to spray complex colloids, wherein the colloid is initially in a substantially liquid form. The electrohydrodynamic method enables sprays or clouds of droplets (“aerosols”) to be produced in which the droplets are monodispersed, that is, they have a very uniform size and do not, unlike some conventional aerosol producing methods, require a propellant gas. This makes inhalers using the electrohydrodynamic method such as described in the applicant's U.S. Pat. No. 4,962,885, U.S. Pat. No. 6,105,877, U.S. Pat. No. 6,105,571, U.S. Pat. No. 5,813,614, U.S. Pat. No. 5,915,377 and WO 99/07478 (which enable delivery of at least partially electrically discharged droplets) and WO 00/35524 (which enables delivery of electrically charged droplets) particularly advantageous because the absence of a gas propellant makes the inhaler easy to use as inhalation does not have to be timed with the expulsion of gas from the inhaler. Further, the monodispersed nature of the aerosol combined with the ability provided by the electrohydrodynamic method to control the size of the droplets enables drugs or other medicaments to be targeted to a particular region of the respiratory system, for example a specific region of the lung. The whole contents of U.S. Pat. No. 4,962,885, U.S. Pat. No. 6,105,877, U.S. Pat. No. 6,105,571, U.S. Pat. No. 5,813,614, U.S. Pat. No. 5,915,377, WO 99/07478 and WO 00/35524 are hereby incorporated by reference. One particular device useful in employing the disclosed methods is described in WO 2007/094835 (Trees et al, filed Aug. 23, 2007) and US 2008/0308095A1, incorporated herein in their entirety.

Using the disclosed methods, bioidentical estrogen, in particular, estradiol, may be delivered so as to achieve regional lung deposition and potential absorption kinetics comparable to IV administration which avoids first-pass hepatic metabolism and the associated effects of first-pass metabolism as seen with conventional oral estrogen products.

In one aspect, the method may comprise administering an estrogen such as bio-identical estradiol to a subject in need of such treatment via the respiratory tract. The method may provide deep-lung deposition and systemic absorption of estrogen comparable to IV administration of estrogen. In one aspect, administration by inhalation is achieved via aerosolization of the composition comprising estrogen with an electrohydrodynamic aerosol device. A suitable device may include the Battelle Mystic™ inhaler as described in WO 2007/094835. In one aspect, the device comprises the Dissociated Discharge EHD Sprayer with Electric Field Shield.

In one aspect, the estrogen delivered via the disclosed methods may be used such that low T. and high C. values are achieved. In one aspect, near 100% bioavailability of the administrated estrogen may be achieved, closely paralleling the effects of intravenous delivery. Accordingly, in one aspect, the disclosed methods may provide greater than about 75%, or greater than about 80%, or greater than about 85%, or greater than about 90%, or greater than about 95%, or about 100% bioavailability, of one or more of the aforementioned estrogens, in particular, estradiol or a biological equivalent thereof

In one aspect, the method may comprise administering estrogen or a biological equivalent thereof, wherein at least two doses are administered within a 24 hour period, such doses being administered at predetermined time points. In this manner, using the EHD device, an estradiol product administered by inhalation at comparable or lower daily doses may achieve a similar PK profile and clinical efficacy when administered one to two times daily while providing a “pulsatile” delivery. Administration of the estrogen may be configured to achieve “peak and trough” serum levels similar to that of a healthy non-menopausal female subject. Such patterns of administration may more closely mirror the more natural estrogen state, and may be associated with a more favorable safety profile based on published literature.

Estrogen therapy administered according to the disclosed methods may be continuous or cyclical. While continuous estrogen therapy alone may be appropriate in women who have undergone a hysterectomy, many clinicians currently recommend that a progestin be added to estrogen therapy in women with an intact uterus. Estrogen alone for 25 or 30 days with addition of progestin therapy for 7 or more days (such as 10 or more days) of a cycle of estrogen administration has been associated with a decreased incidence of endometrial hyperplasia and the attendant risk of endometrial carcinoma in women with an intact uterus. When a progestin is used in conjunction with cyclic estrogen administration, the usual precautions associated with progestin therapy should be observed. Cyclic regimens can lead to a predictable, monthly vaginal bleeding in the initial years of use. Tailored regimens can also be prescribed in a similar way to oral contraceptives, where the regimen is designed to meet the individual needs of the patient. This could include taking estrogen daily and a progestin for a few days every three months. Transdermal patches have a longer dosing schedule and are usually worn for three to four days at a time.

Dosage of estradiol may be individualized according to the condition being treated and the tolerance and therapeutic response of the patient. To minimize the risk of adverse effects, the lowest possible effective dosage may be used. When short-term estrogen therapy is indicated (e.g., for the management of vasomotor symptoms associated with menopause; vulvar and vaginal atrophy), therapy may be discontinued as soon as possible; attempts to reduce dosage or discontinue the drug may be made at 3- to 6-month intervals. Because of the potential increased risk of cardiovascular events, breast cancer, and venous thromboembolic events, estrogen (and combination estrogen/progestin) therapy may be limited to the lowest effective doses and shortest duration of therapy consistent with treatment goals and risks for the individual woman. Estrogen and estrogen/progestin therapy may be periodically reevaluated. Both estrogens and progestogens are available in a range of doses to treat menopausal symptoms. Oral estrogens are generally prescribed at higher doses due the fact that the compounds will pass through the liver. Oral estrogen doses range from 0.1 mg up to 2.5 mg. Transdermal estrogen patch doses range from 0.025 mg to 0.5 mg. There has been growing emphasis on the use of lower dose and transdermally delivered estrogens and progestogens in HRT, both in an attempt to reduce side effects and to potentially lessen safety risks while conferring similar efficacy in reducing vasomotor symptoms and preventing osteoporosis. A number of studies have shown that lower doses of estrogen and combined HRT can prevent bone loss and relieve menopausal symptoms.

In one aspect, the method may comprise administering estrogen at a dose of less than about 25 μg, or less than about 20 μg, or less than about 15 μg, or less than about 10 μg, or less than about 5 μg, or less than about 2.5 μg, or less than about 1.5 μg.

In one aspect, the dose may be administered in a single actuation of said device. In one aspect, the actuation may occur for a period of time of about 2.5 seconds or less, or about 2 seconds or less, or about 1 second or less.

In one aspect, the carrier used in the disclosed methods may comprise ethanol. Without intending to be limited by theory, Applicants believe that the use of ethanol as a carrier may facilitate improved absorption of the active agent, thereby contributing to a rapid systemic effect comparable to IV administration. In one aspect, the composition comprising estrogen may comprise from about 90% to about 99%, or from about 95% to about 98%, or about 95% ethanol, based on total weight of the composition.

In one aspect, the carrier used in the disclosed methods may comprise less than about 10% propylene glycol, or less than about 9% propylene glycol, or less than about 8% propylene glycol, or less than about 7% propylene glycol, or less than about 6% propylene glycol, or less than about 5% propylene glycol, or less than about 4% propylene glycol, or less than about 3% propylene glycol, or less than about 2% propylene glycol, or less than about 1% propylene glycol. In one aspect, the carrier may be substantially free of propylene glycol.

In one aspect, the method may comprise administering, in a single actuation, a spray comprising, based on total number of particles, from about 70% to about 100%, or from about 80% to about 95%, or from about 85% to about 90%, particles having a diameter of less than about 2.5 μm. In one aspect, the method may comprise administering, in a single actuation, a spray comprising, based on total number of particles, from about 70% to about 100%, or from about 80% to about 95%, or from about 85% to about 90%, particles comprise a particle size of from about 0.1 μm to about 2.1 μm, or from about 0.7 μm to about 1.1 μm. Using the disclosed methods, a substantially monodispersed aerosol may be achieved, allowing for single phase delivery similar to a bolus IV injection.

Without being limited by theory, Applicants believe that the disclosed methods allow for a desirable lung deposition profile allowing simulation of IV-like administration as a result of, inter alia, particle size and the use of ethanol as a carrier. Accordingly, in one aspect, the disclosed method may allow for deposition of estrogen into the deep peripheral areas of a lung of a subject in an amount of from about 70% to about 100%, or from about 80% to about 90%, based on total estrogen administered. This allows for direct administration into the cardio-pulmonary circulation. In one aspect, the disclosed method may provide a Tₘₐₓ of from about 1 to about 15 minutes, or from about 2 to about 10 minutes, or from about 4 to about 9 minutes.

In one aspect, the methods provide for delivery of greater than about 50%, or greater than about 60%, or greater than about 70%, or greater than about 75% of aerosol comprising the estrogen to the lungs. In one aspect, greater than about 12%, or greater than about 15%, or greater than about 20%, or greater than about 25% of the estrogen is deposited in the oropharynx region.

Compositions

The compositions comprising estrogen may in some aspects comprise additional components and/or possess additional physical characteristics as described herein.

The compositions disclosed herein may comprise (i) an active ingredient (for example, an estrogen as described above); (ii) a liquid carrier for the active ingredient; (iii) an “aerosol properties adjusting material”; and optionally (iv) additional one or more excipients. The combination of these components may provide a therapeutic composition having enhanced properties for delivery to a user by means of generating an inhalable aerosol.

In one aspect, the composition may comprise a carrier comprising water at a concentration of from about 0% to about 30%, based on total volume of the composition. In one aspect, the composition may comprise a carrier comprising ethanol at a concentration of from about 70% to about 100%, based on total volume of the composition. In one aspect, the composition may comprise a polyol, (such as propylene glycol, PEG-300, or the like) at a concentration of from about 0% to about 30%, based on total volume of the composition. The composition may further comprise an excipient, said excipient being present at a concentration of from about 0% to about 5%, based on total volume of the composition.

In administering an aerosolized active ingredient to the lungs of a patient, considerations include the characteristics of both the composition containing the active ingredient, and the aerosol cloud ultimately be inhaled by the patient or user. The composition may comprise a suitable carrier for the active ingredient such that active ingredient is stable for the desired period of time in the composition; may be consistently sprayable through an aerosol-generating device; and should be designed such that the composition is well-tolerated by the user. The aerosol-generating device itself should effectively and consistently convert the formula into an aerosol cloud, and may have certain desired properties. For example, an aerosol-generating device ideally does not deliver a high velocity aerosol making it difficult for the user to inhale the aerosol particles. Aerosol characteristics may include an aerosol cloud comprising particles that are roughly uniform in size. An aerosol cloud composed of uniform particles of a predetermined size is believed to provide the most efficient and effective delivery of the therapeutic composition to the patient or user because the dosage that the patient receives can be more precisely controlled (i.e., uniform particle size equals more precise delivery and dosage), though in some aspects, particle size may vary or be within a certain range. Aerosols having uniformly-sized particles and uniform distribution patterns are generally desirable over aerosols that do not possess these characteristics because they exhibit more desirable deposition properties within the aerodigestive tract of the user (i.e., they have a higher respirable fraction). When used with compatible compositions, EHD aerosol generating devices can be adjusted to create substantially monomodal aerosols having particles more uniform in size than aerosols generated by other devices or methods. Therefore, for maximum effectiveness of both drug and aerosol device, it is believed that consistent generation of uniformly sized aerosol particles should occur each time the composition is aerosolized with a particular device. The compositions are generally compatible with an aerosol-generating device so that an aerosol cloud with certain preferred properties and characteristics is reproduced each time the device is used.

Physical Characteristics

Liquid compositions that are compatible with EHD aerosol generating devices ideally have characteristics and properties that fall within certain parameters for the aerosol cloud to have the desired properties. In one aspect, relevant physical characteristics of the composition include surface tension, electrical resistivity, and electrical permittivity (dielectric constant). Viscosity of the composition may also be of importance in preparing liquid therapeutic compositions for use with electrostatic or EHD devices.

Surface tension is a property possessed by liquid surfaces whereby these surfaces behave as if covered by a thin elastic membrane in a state of tension. Surface tension is a measure of the energy needed to increase the surface area of the liquid; therefore, liquids with a lower surface tension will aerosolize more easily than liquids with higher surface tension. Surface tension is measured by the force acting normally across unit length in the surface. The phenomenon of surface tension is due to unbalanced molecular cohesive forces near the surface of a liquid. In one aspect, the surface tension of the composition may be within the range of about 10 to 72 milliNewtons/meter. In one aspect, the surface tension of the composition may be within the range of about 15 to 45 milliNewtons/meter. In one aspect, the surface tension of the composition may be within the range of about 20 to 35 milliNewtons/meter.

Electrical conductivity is the ability of a solution to transport electrical charge. The inverse of electrical conductivity is electrical resistivity. Thus, electrical resistivity is a measure of the ability of a material to resist the transport of electrical current, and is a property of a conductor, which gives the resistance in terms of the conductor's dimensions. Liquid compositions with resistivity values of 10 to 100,000 ohm-meters may be aerosolized using EHD aerosol devices, provided that other relevant physical properties are within optimal operating parameters. Thus, in one aspect, the electrical resistivity of the composition may be within the range of about 10 to 100,000 ohm-meters. In one aspect, the electrical resistivity of the composition may be within the range of about 50 to 10,000 ohm-meters. In one aspect, the electrical resistivity of the liquid composition may be within the range of about 200 to 2000 ohm-meters.

Electrical permittivity is a measure of the polarizibility of a liquid, and is relevant in electrostatic spraying processes as it describes the increase in electrical field strength where a fluid is present. To aerosolize solvents with high permittivity (e.g., water), a higher electrical field strength (voltage) is required. It is believed that the permittivity of a liquid composition is not significantly affected by the addition of a small amount (less than 5%) of non-ionic excipients or solvents. In one aspect, the electrical permittivity of the composition may be within the range of about 5 to 500. In one aspect, the electrical permittivity may be within the range of about 10 to 150. In one aspect, the electrical permittivity of the composition may be within the range of about 15 to 50. Electrical permittivity is a dimensionless value denoting the ration of the electrical permittivity of a liquid or material to that of a vacuum.

Viscosity is the measure of the resistance to fluid flow; thus solutions that flow easily generally have lower viscosity. The viscosity of a liquid composition is not affected significantly by the addition of small amounts of drug to the composition. However, the addition of certain suspending agents or very high concentrations of drugs may increase the viscosity of the liquid composition. Viscosity may not be a key solvent parameter in aerosolization of the disclosed compositions using EHD, but it may affect particle size distribution. Highly viscous materials tend to form aerosols with more disperse or bimodal distributions, and with particle sizes larger than desired for respirable aerosols.

Liquid compositions having physical properties within the optimal parameters disclosed above will aerosolize when used with most EHD devices. Controlling the voltage delivered to the system to create the region of high electric field strength also controls the particle size of the aerosol cloud generated by an EHD device. In one aspect, the size of respirable aerosol particles may be from about 0.1 to about 10.0 micrometers. Aerosol particles at the lower end of this range may be required for delivery of the liquid composition to the deep lung, while aerosol particles at the upper end of this range may be required for delivery of the composition to the proximal respiratory tract. For deposition of the composition in the central and peripheral areas of the lung, the aerosol particle size may be about 1.0 to 6.0 micrometers.

Active Ingredient

To benefit the user, the aerosolized liquid composition may comprise at least one additional active ingredient at a concentration permitting delivery of the desired dosage to the patient. The number and types of active ingredients suitable for delivery to a patient by means of an inhaled aerosol varies widely and includes numerous options. In one aspect, the at least one additional active ingredient may comprise any of the following: small molecule and synthetic drugs such as sodium cromoglycate, albuterol sulfate, and triamcinolone acetonide; chemo-therapeutic or chemopreventivc agents such as paclitaxel and doxorubicin; vaccines; nucleic acids, including DNA and RNA vectors and vaccines; aptamcrs, proteins such as insulin; gene therapy agents for treating diseases such as cystic fibrosis; enzymes, hormones; antibodies; vitamins; peptides and polypeptides; oligonucleotides; cells; antigens; allergens; pulmonary surfactant and other surfactants (including synthetic surfactants); anti-infectious agents including antimicrobials, antibiotics, antifungals and antivirals; and pain management drugs such as narcotics.

Preferred initial concentrations of active ingredients in the composition are determined by the required effective dosage of each active ingredient, as well as the efficiency of the pulmonary delivery of the inhaled aerosol. Delivery efficiency and drug efficacy is typically impacted by the selected deposition site within the user's lung.

Carrier Materials

The composition to be aerosolized may also comprise a carrier in which the active ingredient may be dissolved, suspended or emulsified. A variety of solvents or emulsifying agents may be suitable for this purpose. In one aspect, either water or ethanol (depending on the solubility characteristics of the active ingredient) may be used as the solvent in which the active ingredient may be dissolved or suspended. In one aspect, the carrier (solvent) fraction of the composition may represent from about 5 to about 95% of the total volume of the composition. In one aspect, the fraction of the composition represented by the earner may vary depending on the solubility or insolubility of the active ingredient. For example, if an active ingredient is highly soluble in the carrier (e.g. water), then the carrier fraction of the total composition may be as low as about 5% to 10%. If an active ingredient is only moderately soluble in water, a larger fraction of water may be required to completely dissolve or sufficiently suspend the active ingredient.

The pH of desired solvent, as well as the pH of the entire composition, may impact the solubility and stability of the active ingredient. Although pH requirements are likely to differ among specific compositions depending on the active ingredient being used, pH ranges may be in the range of pH about 2 to 9. In one aspect, a pH range of about 3 to 8 is used. In one aspect, a pH range of about 5 to 8 is used.

In one aspect, the solvents selected as carriers may be chosen for use based both on compatibility with certain active ingredients and on their compatibility with EHD devices, and may include water or ethanol. In an alternative embodiment, phospholipids or pulmonary surfactant is used as a carrier. In one aspect, other alcohols such as isopropanol may be employed as carriers. In one aspect, perfluoronated compounds such as perfluorooctanol and perfluorodecalin may be substituted for some or all of the water or ethanol as the carrier material. Such perfluoronated compounds may be useful as alternative carriers for drugs soluble in perfluoronated carriers, micro-suspended medicaments or emulsified mixtures of such pharmaceutical products in water.

Aerosol Properties Adjusting Materials

Certain physical properties of a liquid composition may be critical in enhancing the effectiveness of aerosolization of the composition with an electrostatic or EHD device. Therefore, In one aspect, an aerosol properties adjusting material that provides the desired physical characteristics to the composition represents another possible fraction of the total volume of the liquid composition. In one aspect, the physical properties of the liquid composition may comprise: (i) a surface tension of from about 10 to about 72 milliNewtons/meter; (ii) an electrical resistivity of from about 5 to about 100,000 ohm-meters; and (iii) and an electrical permittivity of from about 5 to about 500. In one aspect, it may be possible to achieve a liquid composition with physical properties falling within these parameters by simply combining the active ingredient and the carrier material. However, if the combination of the active ingredient and the carrier material does not produce a liquid composition having physical properties falling within these parameters, the addition of the aerosol properties adjusting material in amounts that bring the composition within the required parameters may be desired.

In one aspect, the aerosol properties adjusting material may be present in an amount of from about 5% to about 90% of the total volume of the composition. The volume of the aerosol properties adjusting material fraction will vary depending on the volume of the carrier that is required. For example, if the carrier represents 20% of the total volume of the composition, the aerosol properties adjusting material could represent the remaining 80%o of the total volume. The 20/80 volume ratio can apply even with the active ingredient present because the active ingredient may be dissolved in the carrier and/or aerosol property adjusting material. In some instances, the carrier itself may serve as the aerosol properties adjusting material. In one aspect, the aerosol properties adjusting material may be at least one of the following materials or their derivatives; ethanol or other alcohols; propylene glycol; polyethylene glycol; glycerol; oleic acid; medium chain triglycerides; fatty acids; soybean oil; olive oil; phospholipids, and perfluorocarbons. Combinations of these materials may be advantageous in some aspect. For example, the use of ethanol alone may create an aerosol, but the particle size of the aerosol may be below the preferred range. By combining ethanol and polyethylene glycol in a predetermined ratio to one another, the preferred particle size may be achieved.

Excipients

As discussed, there are acceptable ranges of solvent parameters that permit a liquid composition to be aerosolized by the electrohydrodynamic process. Due to the characteristics of certain active ingredients (e.g., ionic, solubility limits, etc.) it may be difficult to formulate a drug at desired concentrations in an appropriate carrier solvent while remaining within the required solvent parameter values. The addition of an excipient can alter a solvent parameter and bring the composition back within the optimal ranges. Addition of an excipient is necessary only where the combined active ingredient, carrier material, and aerosol properties adjusting material do not yield an aerosol with all of the desired characteristics.

In one aspect, the compositions may comprise at least one excipient or a combination of excipients. A broad definition of an excipient is anything in a composition other than an active ingredient. In a more narrow context, an excipient may be added for a variety of purposes including: stabilization of the liquid composition; facilitating control of aerosol particle size; increasing the solubility of the active ingredient in the composition; and lowering the surface tension of the liquid.

Once solubilized, suspended or emulsified, the active ingredient ideally is stable in the carrier itself, and stable in the final composition. In general, as used herein, “stability” means that the active ingredient does not lose activity prior to aerosolization (i.e. retains a reasonable shelf-life), and does not lose activity or degrade significantly as a result of the process of aerosolization. Furthermore, the complete composition is ideally stable over a period of time. In one aspect, stability issues can be addressed by the addition of a stabilizing excipient to the composition.

In one aspect, at least one of the following excipients may be added to increase physical stability of the composition: oils, glycerides, polysorbates, celluloses lecithin, polyvinyl pyrrolidone, polyethyl glycol, saccharide gums, and alginates; while ascorbic acid, citric acid, cyclodextrin, tocopherols or other antioxidants are added to increase chemical stability. In one aspect, chelating or complexing agents such as citric acid, cyclodextrins, and ethylenediaminetetracetic acid may be added to stabilize drug compositions and to increase the solubility of the active ingredient in the composition.

In one aspect, antioxidants such as ascorbic acid and ascorbic acid esters, vitamin E, tocopherols, butylated hydroxyanisole, and butylated hydroxytoluene may be added to reduce degradation of a drug composition caused by oxidation.

An excipient may also be added as a preservative to maintain the microbial integrity of the therapeutic composition. In one aspect, at least one of the following excipients may be added to preserve compositions against microbial contamination or attack: benzalkonium chlorides, phenol, parabens, or any other acceptable antimicrobial or antifungal agent.

By further adjusting physical properties, the addition of excipients may also enhance the overall performance of the composition in terms of the quality of aerosol produced by an EHD device. In one aspect, an ionic compound (e.g., salt) such as sodium chloride, sodium acetate, benzalkonium chloride, or lecithin, may be added to further adjust electrical resistivity, thereby facilitating control of aerosol particle size.

In one aspect, surfactants such as lecithin, polysorbates, poloaxamers, sorbitan esters, glycerides, ethoxylated alcohols, ethoxylated phenols, and ethylene oxide-propylene oxide copolymers may be added to lower the surface tension of the liquid. In one aspect, non-ionic ethoxlyated decyl alcohol (Desonic DA-4) having hydrophilic-lipophilic balance (HLB) of about 10.5 may be added to highly aqueous compositions to enhance the dispersion characteristics of the composition. The use of both pulmonary surfactant and other natural or synthetic surfactants is contemplated herein.

In one aspect, suspending agents such as celluloses, polyvinyl pyrrolidone (povidone or PVP), polyvinyl alcohol (PVA), triglycerides, ethoxylated oils, polyethyl glycol, saccharide gums, and alginates may be added to facilitate suspension of particles, or creation of an emulsion, in a liquid composition. In one aspect, adjuvants such as clove oil, citric acid, caffeine, vaccine adjuvants such as alum, polymers, macromolecules, and oligonucleotides may be added to provide enhanced synergistic efficacy effect between the active ingredient and the excipient. Excipients may also be added to enhance or increase the patient's ability to receive the aerosolized composition. For example, in one aspect, sugars, including sucrose, trehalose, and mannitol, may be added either to stabilize compositions containing proteins, or to serve as sweeteners to improve the taste of the composition. In one aspect, flavoring agents such as sugars, oils, citric acid, menthol, and camphor may be added to improve the flavor of a composition.

### EXAMPLE I

3 mg/mL 17-estradiol in 95% ethanol is adjusted with saline solution in amounts sufficient to adjust the electrical resistivity to about 1200 Ohm-meters or electrical conductivity to about 8.33 microSiemens per centimeter. The composition is then placed in an electrohydrodynamic spray device as described above and the composition is administered to a human subject via nasal inhalation.

All percentages and ratios are calculated by weight unless otherwise indicated.

All percentages and ratios are calculated based on the total composition unless otherwise indicated.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of providing a composition comprising estrogen or a biological equivalent thereof to a subject in need thereof which comprises administering a therapeutically effective amount of said estrogen or a biological equivalent thereof by inhalation, wherein said estrogen or a biological equivalent thereof is administered by way of an electrohydrodynamic device.

2. A method according to claim 1, wherein said estrogen or a biological equivalent thereof is selected from conjugated equine estrogens, estradiol, synthetic conjugated estrogine, micronized estradiol, estropipate, esterified estrogens, salts thereof, and combinations thereof

3. A method according to claim 1, wherein said estrogen or a biological equivalent thereof comprises estradiol or a biological equivalent thereof

4. A method according to claim 1, wherein said estrogen or a biological equivalent thereof comprises 17β-estradiol, or a biological equivalent thereof

5. A method according to claim 1, wherein said composition comprises a progesterone or a biological equivalent thereof

6. A method according to claim 4, wherein said progesterone is selected from progestin, progesterone, medroxyprogesterone, norethindrone, levonorgestral, norethindrone, norgestimate, drospirenone, salts thereof, and combinations thereof

7. A method according to claim 1, wherein said estrogen or a biological equivalent thereof has greater than about 75% bioavailability.

8. A method according to claim 1, wherein said estrogen or a biological equivalent thereof is administered in at least two doses within a 24 hour period at predetermined time points.

9. A method according to claim 7, wherein the administration of at least two doses provides to said subject serum levels having a peak and trough mimicking a healthy non-menopausal female subject.

10. A method according to claim 1, wherein said estrogen or a biological equivalent thereof comprises estradiol, said estradiol being administered at a dose of less than about 25 μg.

11. A method according to claim 10, wherein said dose is administered in a single actuation of said device.

12. A method according to claim 11, wherein said actuation occurs for a period of time of about 2.5 seconds or less.

13. A method according to claim 1 wherein said carrier comprises ethanol.

14. A method according to claim 3, wherein said composition comprises from about 90% to about 99% ethanol, based on total weight of said composition.

15. A method according to claim 1, wherein said composition comprises less than about 10% propylene glycol.

16. A method according to claim 1, wherein said electrohydrodynamic device, in a single actuation, produces a spray comprising from about 70% to about 100% particles having a diameter of from about 0.1 μm to about 2.5 μm.

17. A method according to claim 1, wherein from about 70% to about 100% of said estrogen is deposited into the deep peripheral areas of a lung of said subject.

18. A method according to claim 1, wherein said estrogen is administered directly into the cardio-pulmonary circulation.

19. A method according to claim 1, wherein said method provides a Tₘₐₓ of from about 1 to about 15 minutes.
